(19) United States
(12) Patent Application Publication
(10) Pub. No.: US 2011/0256596 A1
(43) Pub. Date: Oct. 20, 2011

(54) MANUFACTURING METHOD OF OPTICALLY ACTIVE D TYPE LACTIC ACID USING BYPRODUCTS PREPARED FROM RICE POLISHING PROCESS

(75) Inventors: Chae Hwan Hong, Ansan (KR); Do Suck Han, Seongnam (KR)

(73) Assignee: HYUNDAI MOTOR COMPANY, Seoul (KR)

(21) Appl. No.: 12/829,083

(22) Filed: Jul. 1, 2010

(30) Foreign Application Priority Data
Apr. 20, 2010 (KR) 10-2010-0036559

(51) Int. Cl. C12P 7/56

(52) U.S. Cl. 435/139

(57) ABSTRACT

Disclosed is a method for manufacturing D type lactic acid wherein byproducts (e.g., rice bran and pulverized rice) obtained during a rice polishing process are saccharified by using α-amylase and amyloglucosidase and then fermented by a microorganism, Sporolactobacillus inulinus. According to the method, byproducts generated during a rice polishing process can be used to produce D type lactic acid that can be used as a material for the manufacture of D-type polylactic acid, which is useful for the manufacture of a stereo complex of polylactic acid, a suitable plant-derived material for the manufacture of materials interior and exterior parts of vehicles.

### CROSS-REFERENCE TO RELATED APPLICATION

This application claims under 35 U.S.C. §119(a) the benefit of Korean Patent Application No. 10-2010-0036559 filed Apr. 20, 2010, the entire contents of which are incorporated herein by reference.

### BACKGROUND

(a) Technical Field

The present disclosure relates to a method of manufacturing D type lactic acid using byproducts such as rice bran and pulverized rice obtained during a rice polishing process. The D type lactic acid manufactured in the present invention can be used as a polylactic acid material essential for the manufacture of stereocomplex polylactic acid thus rendering improved heat resistance and impact resistance on the polylactic acid manufactured therefrom.

(b) Background Art

Studies have been focused on developing eco-friendly energy sources. One of such eco-friendly energy sources is a plant. A plant-derived polymer, a biomass polymer, is a material manufactured from recyclable plant resources such as corns, beans, sugarcanes, beets, woods or the like, via a chemical or biological method. It is known more effective than biodegradable materials in coping with environmental issues such as carbon dioxide reduction. Of biomass polymers, polylactic acid or polylactide (PLA) is a linear aliphatic polyester. It can be obtained by fermentation of starch from corn or potato, or by polymerization of sugar monomers obtained by fermentation after saccharification of plant cellulose. It has been known as a carbon-neutral and eco-friendly thermoplastic polymer material.

However, these PLA materials are inferior to those of general polymer materials in physical properties thus having a very limited range of industrial application. In particular, heat and impact resistance of the PLA materials is not sufficient for the PLA materials to be used for materials for the manufacture of vehicles. A known method to resolve the above drawback is to form a stereo complex by blending L type stereoisomeric polylactic acid (PLLA) with D type stereoisomeric polylactic acid (PDLA). So far, the global manufacture of PLA is largely limited to PLLA and there is only a very limited amount of production of PLDA. This is because D- or L-type lactides, essential for the manufacture of PDLA, cannot be effectively manufactured in a large scale.

Korean Patent Application Publication No. 10-2010-0005820 discloses a method of selectively manufacturing one type of lactide with equal optical activity from optically unpure lactic acid or alkyl lactate mixture. However, this method has an disadvantage requiring an additional process for the separation of lactic acid which did not react with selectively synthesized lactide or alkyl lactate. Further, Korean Patent Nos. 10-0122428 and 10-0308521 disclose methods to selectively manufacture D type lactic acid by using transformed E. coli. However, this also has a disadvantage in large scale production requiring a genetic pretreatment to obtain transformed E. coli.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention has been made in an effort to solve the above-described problems associated with prior art.

One of the objects of the present invention is to provide a method of manufacturing D type lactic acid with high productivity and yield in a cost effective.

In an embodiment, the present invention relates to a method of manufacturing D type lactic acid comprising: preparing a rice slurry by pulverizing byproducts obtained from a rice polishing process; preparing a first mixture solution by adding α-amylase to said rice slurry and conducting an enzyme reaction; preparing a second mixture solution by adding amyloglucosidase to the first mixture solution and conducting an enzyme reaction; apoptosizing the enzyme activity of the second mixture solution and separating hydrolysate therefrom; and inoculating the hydrolysate with Sporolactobacillus inulinus and conducting fermentation.

According to the method of manufacturing D type lactic acid of the present invention, about 100 g of D type lactic acid can be promptly manufactured per 1000 g of byproducts obtained during a rice polishing process. Thus manufactured D type lactic acid can be used as a material for the manufacture of D type polylactic acid, which can be then used as materials for the synthetic body materials, optically active herbicide or the like, in particular, for the manufacture of stereo complex of polylactic acid.

The above and other features of the present invention will be described below.

### DETAILED DESCRIPTION

Hereinafter reference will now be made in detail to various embodiments of the present invention, examples of which are described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The present invention relates to a method for manufacturing D type lactic acid wherein byproducts (e.g., rice bran and pulverized rice) obtained during a rice polishing process are saccharified by using α-amylase and amyloglucosidase and then fermented by using a microorganism, Sporolactobacillus inulinus.

The above slurry of rice byproducts is prepared by pulverizing the rice byproducts of rice bran and pulverized rice into fine powder and then mix with tap water.

The rice to be used in the present invention is not limited to certain species but any rice species including ‘Akibari’, ‘Saechucheong’, ‘Odae’, ‘Hitomebore’, and ‘Junam’ may be used. The rice byproduct and water are mixed preferably in a 1:2 volume ratio. After rice byproduct slurry is prepared, its pH is adjusted to pH 5.5-6.0, added with α-amylase, saccharified by enzyme reaction, to prepare a first mixed solution. If the pH is below 5.5 or greater than 6.0, the hydrolytic activity of α-amylase may be deteriorated and thus it is preferred that the above range be maintained.

As for the α-amylase, it is preferred that its activity unit is in the range of 18,000-23,000 U/cc. It is preferably added 12-14 U per 1 g of rice byproducts. If α-amylase is added less than 12 U, its enzyme activity may not be sufficient. Meanwhile, if α-amylase is added more than 14 U, it will not be cost-effective. Thus it is preferred that the above range be maintained.

Preferably, saccharification by α-amylase is conducted at 90˜100° C. for 50˜70 minutes. If the saccharification is conducted at a temperature below 90° C., it will decrease the saccharification efficiency. Meanwhile, if the saccharification is conducted at a temperature above 100° C., it will reduce the saccharification efficiency due to thermal distortion. Further, if the saccharification is conducted for less than 50 minutes, it may result in insufficient saccharification. Meanwhile, if the saccharification is conducted for more than 70 minutes, saccarification efficiency may not improve. Therefore, it is preferred that the above range be maintained.

The first mixture solution prepared by using α-amylase is cooled down to 60° C. or below, adjusted to pH 4.0˜4.5, added with amyloglucosidase, and saccharified by enzyme reaction to obtain the second mixture solution. Preferably, amyloglucosidase having its activity unit in the range of 15,000˜20,000 U/cc is used. Amyloglucosidase is added in the range of 110˜130 U based on 1 g of rice byproducts. If it is added less than 110 U, its enzyme activity may not be sufficient. Meanwhile, if it is added more than 130 U, it will not be cost-effective. Therefore, it is preferred that the above range be maintained.

Preferably, saccharification by amyloglucosidase is preferably conducted at 55˜60° C. for 25˜35 hours. If the saccharification is conducted at a temperature below 55° C., saccharification may not be sufficient. Meanwhile, if the saccharification is conducted at a temperature above 60° C., it will reduce the saccharification efficiency due to thermal distortion. Further, if the saccharification is conducted for less than 25 hours, it may result in insufficient saccharification. Meanwhile, if the saccharification is conducted for more than 35 hours, saccarification efficiency may not improve. Therefore, it is preferred that the above range be maintained.

Upon completion of the saccharification by using amyloglucosidase, the second mixture solution is increased to 100° C. or above, maintained thereat for at least 10 minutes thereby apoptosizing enzyme activity. Then, the second mixture solution is cooled down to room temperature, and hydrolysate and the remaining solid sludge are separated by ultracentrifugation. Finally, the sugar concentration of the hydrolysate is preferably in the range of 80˜130 g/L. In case the sugar concentration of the rice byproducts is lower than 80 g/L, starch particles may be added to increase sugar concentration.

Then, the above hydrolysate is inoculated with Sporolactobacillus inulinus to conduct D type lactic acid fermentation. Here, it is preferred that the hydrolysate is sterilized to get rid of any microorganisms invaded during the saccharification process before it is inoculated with Sporolactobacillus inulinus, for example, by exposing at 120° C. for 20 minutes.

As for the Sporolactobacillus inulinus, a seed culture wherein Sporolactobacillus inulinus is cultured in a medium kept at 40˜45° C. for 24˜36 hours is used. Preferably, the amount of the seed culture to inoculate the hydrolysate is in the range of 5˜10 volume %. If the amount of inoculation is less than 5 volume %, it will lower the fermentation process. Meanwhile, if the amount of inoculation is greater than 10 volume %, it will not be cost-effective. Fermentation reaction is conducted in an anaerobic condition with oxygen concentration of 0.1˜0.2 volume % at 40˜45° C. to produce D type lactic acid. If the reaction temperature is above 45° C. or below 40° C., the resulting yield will become lowered.

As for the pH condition of the mixture solution, it is preferable to keep it in the range of pH 4.0-5.0. If the pH is lower than 4.0 or higher than 5.0, it will result in the decrease in the yield of product. Therefore, it is preferred that the above range be maintained.

The D type lactic acid manufactured by using Sporolactobacillus inulinus can be separated by liquid chromatography and recovered thereafter, but is not limited thereto. According to the method of manufacturing D type lactic acid of the present invention, about 100 g of D type lactic acid can be produced per 1,000 g of rice byproducts. The reaction rate of the present invention is about 1.3 g/L·h thereby providing an excellent productivity. Thus prepared D type lactic acid can be used as monomers of D type polylactic acid.

### EXAMPLES

The following examples illustrate the invention and are not intended to limit the same.

### Example

Rice byproducts such as rice bran and pulverized rice generated during a rice polishing process were pulverized into fine powder, and then mixed with tap water in a 1:2 volume ratio to prepare a rice slurry. The rice slurry was added with CaCl₂ to adjust its pH to 6.0. The resultant was added with α-amylase (Wuxi Jieneng Bioengineering, China), a hydrolytic enzyme having an active unit of 20,000 U/cc, at a concentration of 14 U per 1 g of rice byproducts, and kept at 95° C. for 60 minutes to prepare the first mixture solution. Then, the first mixture solution was cooled down to 60° C., lowered its pH to 4.5 by adding CaCl₂, added with amyloglucosidase (Wuxi Jieneng Bioengineering, China), at a concentration of 110 U per 1 g of rice byproducts, and kept at 60° C. for 30 hours to prepare the second mixture solution. The second mixture solution was heated to 100° C., maintained thereat for 10 minutes thereby apoptosizing the enzyme activity. The second mixture solution was then cooled to room temperature, and the hydrolysate and the remaining solid slurry were separated by ultracentrifugation. Thus obtained hydrolysate was analyzed by HPLC(High Performance Liquid Chromatography) and revealed that its sugar concentration was 100 g/L.

Sporolactobacillus inulinus (ATCC1553) was cultured in a medium, kept at 40˜45° C., comprising 10.0 g of pancreatic digest of gelatin, 8.0 g of beef extract, 20.0 g of dextrose, 2.0 g of dipotassium phosphate, 1.0 g of polysorbate 80, 5.0 g of sodium acetate, 2.0 g of ammonium citrate, 0.2 g of magnesium sulfate, and 0.05 g of manganese sulfate for 24˜36 hours to prepare a seed culture. The above hydrolysate was sterilized at 120° C. for 20 minutes. Its pH was adjusted to 4 by using CaCO₃, inoculated with the above seed culture at a concentration of 5 volume %, fermented at 42° C. to produce D type lactic acid. The change in the concentration of D type lactic acid was analyzed by HPLC. The result is shown in the Table 1 below.

### Comparative Examples 1-2

D type lactic acid was produced in the same manner as in the above Example, except that Lactobacillus bulgaricus (DSM2129) and Lactobacillus sp. RKY2 (KCTC 10353BP) were used in Comparative Example 1 and Comparative Example 2, respectively, instead of Sporolactobacillus inulinus (ATCC1553).

TABLE 1

| | Conc. of D-Type Lactic Acid (g/L) | | |
|---|---|---|---|
| | | Comparative | Comparative |
| Time (hr) | Example | Example 1 | Example 2 |
| 0 | 0 | 0 | 0 |
| 10 | 19 | 15 | 14 |
| 20 | 22 | 17 | 18 |
| 30 | 28 | 20 | 21 |
| 40 | 46 | 29 | 30 |
| 50 | 54 | 37 | 36 |
| 60 | 70 | 45 | 44 |

As shown in Table 1 above, in Example 1, the concentration of D type lactic acid reached 70 g/L after 60 hours of culture. This concentration was much higher than the concentration of D type lactic acid obtained in Comparative Examples 1 and 2. Accordingly, the present method is shown to produce D type lactic acid in high yield, and enables a large scale production of D type polylactic acid monomers in a cost-effective way.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A method of manufacturing D type lactic acid comprising:
preparing a rice slurry by pulverizing byproducts obtained from rice polishing process;
preparing a first mixture solution by adding α-amylase to said rice slurry and conducting an enzyme reaction;
preparing a second mixture solution by adding amyloglucosidase to said first mixture solution and conducting an enzyme reaction;
apoptosizing the enzyme activity of said second mixture solution and separating hydrolysate therefrom; and
inoculating said hydrolysate with Sporolactobacillus inulinus and conducting fermentation.

2. The method of manufacturing D type lactic acid according to claim 1, wherein said enzyme reaction by α-amylase is conducted at 90-100° C. under pH 5.5-6.0 for 50-70 minutes.

3. The method of manufacturing D type lactic acid according to claim 1, wherein said enzyme reaction by amyloglucosidase is conducted at 55-60° C. under pH 4.0-4.5 for 25-35 hours.

4. The method of manufacturing D type lactic acid according to claim 1, wherein said fermentation reaction by Sporolactobacillus inulinus is conducted at 40-45° C. under pH 4.0-5.0.
